SEQUENCE LISTING (11 sequences)

SEQ ID NO: 1 - protein, 338 aa, human herpesvirus 1
MLAVRSLQHLSTVVLITAYGLVLVWYTVFGASPLHRCIYVVRPTGTNNDTALVWMKMNQTLLFLGAPTHPPNGGWRNHAHISYANLIAGRVVPFQVPPDAMNRRIMNVHEAVNCLETLWYTRVRLVVVGWFLYLAFVALHQRRCMFGVVSPAHKMVAPATYLLNYTGRIVSSVFLQYPYTKITRLLCELSVQRQNLVQLFETDPVTFLYHRPAIGVIVGCELIVRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 2 - protein, 300 aa, human herpesvirus 1
MLAVRSLQHLSTVVLITAYGLVLVWYTVFGHPPNGGWRNHAHISYANLIAGRVVPFQVPPDAMNRRIMNVHEAVNCLETLWYTRVRLVVVGWFLYLAFVALHQRRCMFGVVSPAHKMVAPATYLLNYTGRIVSSVFLQYPYTKITRLLCELSVQRQNLVQLFETDPVTFLYHRPAIGVIVGCELIVRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 3 - protein, 35 aa, Artificial Sequence
  Coiled Coil Peptide
EVSALEKEVSALEKEVSALEKEVSALEKEVSALEK

SEQ ID NO: 4 - protein, 35 aa, Artificial Sequence
  Coiled Coil Peptide
KVSALKEKVSALKEKVSALKEKVSALKEKVSALKE

SEQ ID NO: 5 - protein, 32 aa, Artificial Sequence
  Coiled Coil Peptide
VAQLRERVKTLRAQNYELESEVQRLREQVAQL

SEQ ID NO: 6 - protein, 32 aa, Artificial Sequence
  Coiled Coil Peptide
VDELQAEVDQLQDENYALKTKVAQLRKKVEKL

SEQ ID NO: 7 - protein, 38 aa, Artificial Sequence
  Protein A IgG Binding Domain
AVAQSFNMQQQRRFYEALHDPNLNEEQRNAKIKSIRDD

SEQ ID NO: 8 - protein, 33 aa, Artificial Sequence
  Protein A IgG Binding Domain
FNMQQQRRFYEALHDPNLNEEQRNAKIKSIRDD

SEQ ID NO: 9 - DNA, 3670 bp, human herpesvirus 1
atgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgcccccccgcgccaccctcactgcccgtcgcgcgtgtttgatgttaataaataacgcataaatttggctggttgtttgttgtctttaatggaccgcccgcagggggggtggcatttcagtgtcgggtgacgagcgcgatccggccgggagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcatgtacaggatgcaactcctgtcttgcattgcactaagtcttgcacttgtcacgaattcgatatcgacagcccacccctctcctagcccaagatccgccggccagttcgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgagtggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcctaggaccccaaaagtttgtctgcgtattccagggcggggctcagttgaatctcccgcagcacctctaccagcaggtccgcggtgggctggagaaactcggccgtcccggggcaggcggtcgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacat

SEQ ID NO: 10 - DNA, 6903 bp, human herpesvirus 1
ttagaggcgccgggagtggggtcgtcggcgtctgctttttgtggcggcgtcccgtcgcggggtggggtccgacgtggcgatgatgggcggcggcgtggtgaggggcttcggctgcaggcccgcttcatcgtccggcggcagaaccggggtccgtccagacgttccgttggtaggtcccaaatcctgtcgccctacacagcggcgggtgcgcgaatagtcaaagttcacacacccagccttcacaggtgtgtggctggcggcctgcttgcgactgtccagcgcctggcgtatctctttataaagggccaagcgcgtttctgtttcctgggtgttggcaggaaacgcggggtaactcaagtcctccaaaaaacccgccacaaataaaaaggggttaacccaatatgccttctgggcatgcctatcccacaagaccgtgtccaatccgggacagtgataacgcaaaaatataccgtctatcaaagcatagtttatagccgagggggtctcgtaacgccaatcaagatcgtcagacgggagcggcacacaaggcacctttaatatatcccccacctctcgagccacccgactccgaataacatattcggttgaagacaagcccccccgcacacacaaaaccccaacggcaataagcccgacccaacccaaaatccccatagcgcctagggtcggcacccacagaaacctacagtccccaagtgtttgcccagtaacacaaccacgacgtcgtgccacacaagccccgtatccccgttcccgcgcttttcgttggtttatatacacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcgccaccatgtgccatcagcagctggtcatctcatggttctccctggtgtttctggcctcacctctggtcgcaatctgggaactgaaaaaggatgtgtacgtggtggagctggactggtatcccgatgcccctggcgagatggtggtgctgacctgcgacacacccgaggaggatggcatcacctggacactggatcagagctccgaggtgctgggaagcggcaagaccctgacaatccaggtgaaggagttcggcgacgccggccagtacacctgtcacaagggaggagaggtgctgagccactccctgctgctgctgcacaagaaggaggatggcatctggtccacagacatcctgaaggatcagaaggagccaaagaacaagaccttcctgcggtgcgaggccaagaattatagcggccggttcacctgttggtggctgaccacaatctccaccgatctgacattttctgtgaagtctagcaggggatcctctgacccacagggagtgacatgcggagcagccaccctgagcgccgagagggtgcgcggcgataacaaggagtacgagtattccgtggagtgccaggaggactctgcctgtccagcagcagaggagtccctgcctatcgaagtgatggtggatgccgtgcacaagctgaagtacgagaattataccagctccttctttatccgggacatcatcaagcccgatccccctaagaacctgcagctgaagcctctgaagaatagcagacaggtggaggtgtcctgggagtaccctgacacctggagcacaccacactcctatttctctctgaccttttgcgtgcaggtgcagggcaagtccaagcgggagaagaaggacagagtgttcaccgataagacatctgccaccgtgatctgtagaaagaacgcctctatcagcgtgagggcccaggaccgctactattctagctcctggtccgagtgggcctctgtgccttgcagcggcggaggaggaggaggatctatgtgcccagcaaggagcctgctgctggtggccacactggtgctgctggatcacctgtccctggcaaggaatctgccagtggcaacccctgacccaggcatgttcccctgcctgcaccacagccagaacctgctgagggccgtgtccaatatgctgcagaaggcccgccagacactggagttttacccttgtaccagcgaggagatcgaccacgaggacatcacaaaggataagacctccacagtggaggcctgcctgccactggagctgaccaagaacgagtcctgtctgaacagccgggagacaagcttcatcaccaacggctcctgcctggcctctagaaagacaagctttatgatggccctgtgcctgtctagcatctacgaggacctgaagatgtatcaggtggagttcaagaccatgaacgccaagctgctgatggaccccaagaggcagatctttctggatcagaatatgctggccgtgatcgacgagctgatgcaggccctgaacttcaatagcgagacagtgcctcagaagtcctctctggaggagccagatttctacaagaccaagatcaagctgtgcatcctgctgcacgcctttcggatcagagccgtgacaatcgaccgcgtgatgtcctatctgaacgcctctggaggaggaggaagcggaggaggaggatccggcggcggcggctccggctctggcgccaccaacttctccctgctgaagcaggcaggcgacgtggaggagaatccaggacctatgcggatctctaagcctcacctgagaagcatctccatccagtgctacctgtgcctgctgctgaacagccactttctgacagaggccggcatccacgtgttcatcctgggctgttttagcgccggcctgccaaagaccgaggcaaactgggtgaatgtgatctctgacctgaagaagatcgaggatctgatccagagcatgcacatcgatgccacactgtataccgagtctgacgtgcaccctagctgcaaggtgaccgccatgaagtgtttcctgctggagctgcaggtcatcagcctggagtccggcgacgcaagcatccacgatacagtggagaacctgatcatcctggccaacaatagcctgagctccaacggcaatgtgaccgagtccggctgcaaggagtgtgaggagctggaggagaagaacatcaaggagttcctgcagagctttgtgcacatcgtgcagatgttcatcaatacatccggaggaggaggatcaggcggaggaggaagcggcggcggcggctctggcagcggcgccaccaacttttccctgctgaagcaggccggcgatgtggaagaaaatccaggaccaatggcaccaaggagagcaaggggatgcagaaccctgggcctgcctgccctgttattactgctgctgctgaggccaccagcaacaaggggaatcacctgtcctccacccatgagcgtggagcacgccgacatctgggtgaagtcctactctctgtattctagggagcggtacatctgcaacagcggctttaagaggaaggccggcacatctagcctgaccgagtgcgtgctgaacaaggccaccaatgtggcccactggaccacaccttccctgaagtgcatcagggatccagccctggtgcaccagcgccccgcacctccaagcacagtgaccacagcaggagtgacccctcagccagagagcctgtccccttctggcaaggagccagcagcatcctctccaagctccaacaatacagcagcaaccacagcagcaatcgtgccaggctcccagctgatgccaagcaagtccccctctaccggcaccacagagatctctagccacgagtcctctcacggcacaccaagccagaccacagccaagaattgggagctgaccgcaagcgcctcccaccagccacctggcgtgtacccacagggacactccgacaccacagtggccatctctaccagcacagtgctgctgtgcggcctgtctgccgtgagcctgctggcctgttatctgaagtccaggcagaccccaccactggcatctgtggagatggaggccatggaggccctgcccgtcacatgggggactagtagtagagacgaggatctggaaaactgtagtcaccatctgtaagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatccactagttctagagcggccgccgcacataaaggcccggcgcgaccgacgcccgcagacggcgccggccacgaacgacgggagcggctgcggagcacgcggaccgggagcgggactcgcagagggccgtcggagcggacggcgtcggcatcgcgacgccccggctcgggatcgggatcgcatcggaaagggacacgcggaaagacccacccaccccacccacgaaacacaggggacgcaccccgggggcctccgacgacagaaacccaccggtccgcctttgtgcacgggtaagcaccttgggtgggcggaggaggggggacgcgggggcggaggagggggctcacccgcgttcgtgccttcccgcaggaggaacgtcctcgtcgaggcgaccggcggcgaccgttgcgtggaccgcttcctgctcgtcggggcgaccggcggcgaccgttgcgtggaccgcttcctgctcgtcgggcgggggagcatgtcgtgggccctggaaatggcggacaccttcctggacaacatgcgggttgggcccaggacgtacgccgacgtacgcgatgagatcaataaaagggggcgtgaggaccgggaggcggccaaaaccgccgtgcacgacccggagcgtcctctgctgcgctctcccgggctgctgcccaaaatcgcccccaacgcatccttgggtgtggcacatcgaagaaccggcgggaccgtgaccgacagtccccgtaatccggtaacccgttgagtcccgggtacgaccatcgcccaagtttctgggcggagggtggttccccccgtggctctcgagatgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccccgggagccccggcctccccgggagccccggaccccacgcaccccccgcgaaccacgtacggctcgcgggtctgtatagcccgggcaagtatgcccccctggcgagcccagaccccttctccccacaagatggagcgtacgctcgggcccgcgtagggctccacaccgcggttcgcgtcccgcccaccggaagcccaacccacacgcacttgcggcatgacccgggcgatgagccaacctcggatgactcagggctctaccctctggacgcccgggcgcttgcgcacctggtgatgttgcccgcggaccaccgggccttctttcgaaccgtggtcgaggtgtctcgcatgtgcgctgcaaacgtgcgcgatcccccgcccccggctacaggggccatgttgggccgccacgcgcggctggtccacacccagtggctccgggccaaccaagagacgtcgcccctgtggccctggcggacggcggccattaactttatcaccaccatggccccccgcgtccaaacccaccgacacatgcacgacctgttgatggcctgtgctttctggtgctgtctgacacacgcatcgacgtgttcgtacgcggggctgtactcgacccactgcctgcatctgtttggtgcgtttgggtgtggggacccggccctaaccccacccctgtgctag

SEQ ID NO: 11 - DNA, 3578 bp, human herpesvirus 1
atgctcgccgtccgttccctgcagcacctctcaaccgtcgtcttgataacggcgtacggcctcgtgctcgtgtggtacaccgtcttcggtcacccccccaacgggggctggcgcaaccacgcccatatctgctacgccaatcttatcgcgggtagggtcgtgcccttccaggtcccacccgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcaatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcaaagcttatgatagcatcttgttcttagtctttttcttaatagggacataaagcccacaaataaaaatatgcctgaagaatgggacaggcattgggcattgtccatgcctagtaaagtactccaagaacctatttgtatactagatgacacaatgttctagccaagcttggtagtcatcatgtttaaacatctaccattccagttaagaaaatatgatagcatcttgttcttagtctttttcttaatagggacataaagcccacaaataaaaatatgcctgaagaatgggacaggcattgggcattgtccatgcctagtaaagtactccaagaacctatttgtatactagatgacacaatgtcaatgtctgtgtacaactgccaactgggatgcaagacactgcccatgccaatcatcctgaaaagcagctataaaaagcaggaagctactctgcaccttgtcagtgaggtccagatacctacagagggatccactagttctagtcgaggtcgaggcaccccattcctggcctctgtctcccgcaccctatcccttcacagcctgtgctctaggggactggagatttccaaaacctgacccgatccctccccagttcagttccttctactgctttgggtggaaggctggtcgttgtgttaaaaggcaggaagggagaaagttgcatttaaactttaggagctgcgtcacggcagtctcctggagaaagctccgccgaacgggacagattctttttgcaacttggaggcgccgggcgtggggaggaggcggcgcgcggggcgggggcgcgcggggccggggtgcaggcggggacgcggggtgacgcgggcccgggccgctgtagcacacaggggctcggtctctcggcttcaggcggagtccggctgcactaggctgggagcgcggcgggacgcgaaccgggaggctggcagcccgcgggcgagccgcgctggggggccgaggccggggtcggggccggggagccccgagagctgccgcagcggggtcccggggccgcggaggggccatggctgccggcagcatcacttcgcttcccgcactgccggaggacggcggcggcgccttcccacccggccacttcaaggatccgtcatggcgactgacattgatatgctaattgacctcggcctggacctctccgacagcgatctggacgaggacccacccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgcgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgttggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacacaccgccaggagtgttcgagtcgtgtctgcgagttgacggccagtcacatcgtcgcccccccgtacgtgcacggcaaatatttttattgcaactccctgttttag

(19) United States
(12) Patent Application Publication
(10) Pub. No.: US 2017/0319639 A1
(43) Pub. Date: Nov. 9, 2017

(54) ONCOLYTIC VIRUSES & METHODS OF USE THEREOF

(71) Applicant: William Jia, Vancouver (CA)

(72) Inventor: William Jia, Vancouver (CA)

(21) Appl. No.: 15/588,616

(22) Filed: May 6, 2017

Related U.S. Application Data
(60) Provisional application No. 62/333,036, filed on May 6, 2016.

(51) Int. Cl. A61K 35/763; C07K 14/005; C12N 7/00

(52) U.S. Cl. CPC A61K 35/763; C12N 7/00; C07K 14/005; C12N 2710/16622; C12N 2710/16632; C12N 2710/16621

(57) ABSTRACT

Modified and improved oncolytic viruses (and methods of use thereof) are disclosed. More particularly, modified and oncolytic human herpesviruses (and methods of use thereof) are disclosed, which include a modified amino acid sequence that includes a deletion in a region that represents the amino terminus of glycoprotein K. The modified and oncolytic human herpesviruses include a deletion of amino acid residues 31-68, relative to the wild type amino acid sequence of glycoprotein K in human herpesviruses. Isolated vector constructs that encode such oncolytic viruses are also disclosed. In addition, methods for using such oncolytic viruses to treat cancers are disclosed.

### CROSS-REFERENCE TO RELATED APPLICATIONS

This application claims priority to, and incorporates by reference, U.S. provisional patent application Ser. No. 62/333,036, filed on May 6, 2016.

### FIELD OF THE INVENTION

The field of the present invention relates to certain modified and improved oncolytic viruses and methods of use thereof. More particularly, the field of the present invention relates to certain modified and oncolytic human herpesviruses and methods of use thereof.

### BACKGROUND OF THE INVENTION

Today, cancer is primarily treated using conventional therapies, such as surgery, chemotherapy, and radiation. Surgery is typically used as the primary treatment for early stages of cancer; however, many tumors cannot be completely removed by surgical means. In addition, metastatic growth of a cancer may prevent the complete cure of cancer by surgery. Chemotherapy involves administration of compounds having antitumor activity, such as alkylating agents, antimetabolites, and antitumor antibiotics. However, the efficacy of chemotherapy is often limited by severe side effects, including nausea and vomiting, bone marrow depression, renal damage, and central nervous system depression. Radiation therapy relies on the greater ability of normal cells, in contrast to cancer cells, to repair themselves after treatment with radiation. Radiotherapy cannot be used to treat many cancers, due to the sensitivity of tissue surrounding the tumor. In addition, certain tumors have demonstrated resistance to radiotherapy.

In recent years, oncolytic viruses have offered a potentially new, effective, and less toxic way of treating cancers. However, drawbacks continue to exist with oncolytic viruses as well, such as inadequate efficacy and/or undesirable off-target effects. In view of the drawbacks associated with the current means for treating cancers (including currently-available oncolytic viruses), a continued need exists for new and improved oncolytic viruses, which are less toxic to a subject and yet effective for the treatment of cancer. As the following will demonstrate, the oncolytic viruses (and methods of use thereof) of the present invention address such demands, as described further below.

### SUMMARY OF THE INVENTION

According to certain aspects of the present invention, modified and improved oncolytic viruses (and methods of use thereof) are provided. More particularly, the invention encompasses certain modified and oncolytic human herpesviruses (and methods of use thereof), which include a modified amino acid sequence that comprises a deletion in a region that represents the amino terminus of glycoprotein K. More specifically, the modified oncolytic human herpesviruses of the present invention include a deletion of amino acid residues 31-68, relative to the wild type amino acid sequence of glycoprotein K in human herpesviruses.

According to certain related aspects of the invention, the oncolytic human herpesviruses described herein (which include a deletion of amino acid residues 31-68, relative to the wild type amino acid sequence of glycoprotein K in human herpesviruses) may be co-expressed or co-delivered with, for example, growth factors and/or cytokines that are effective to stimulate a subject's immune system (e.g., interleukins, such as IL12 or IL15; an IL15 receptor alpha unit; interferons; or combinations of the foregoing). In addition, according to certain aspects of the invention, the oncolytic human herpesviruses may be co-expressed or co-delivered with checkpoint inhibitors—e.g., proteins that disrupt the interaction of PD-1 and proteins expressed on the surface of cancer cells known as PD-L1 and PD-L2.

According to certain additional aspects of the invention, the viral envelope of the modified oncolytic human herpesviruses may be decorated with an antibody, antibody fragment, or polypeptide that is capable of binding to a protein expressed on a cell surface of a cancer cell, which is effective to selectively target the oncolytic human herpesviruses to cancer cells (and preferably avoid undesirable off-target effects, damage to normal cells, and toxicity in a subject). In certain embodiments, the antibody, antibody fragment, or polypeptide may be tethered to the viral envelope through coiled coil peptide interactions. In other embodiments, when the viral envelope is decorated with an antibody (or certain antibody fragments), the antibody (or antibody fragments) may be tethered to the viral envelope through interactions between the Fc domain thereof and a Protein A IgG binding domain.

According to additional aspects of the present invention, isolated vector constructs that encode a modified human herpesvirus described herein are provided.

According to yet further aspects of the invention, methods of using the oncolytic viruses (and isolated vector constructs) described herein to treat cancers are also provided, which are particularly useful for treating non-central nervous system (CNS) solid tumors.

The oncolytic human herpesviruses, isolated vector constructs, and methods of use thereof of the present invention are described in further detail below, in the Detailed Description of the Invention section.

### BRIEF DESCRIPTION OF THE FIGURES

FIG. 1 is a bar graph showing the effects of the modified and oncolytic human herpesviruses disclosed herein on various types of cancer cells, including human colon cancer cells (CT26), human lung cancer cells (H460), mouse lung cancer cells (LL2), human glioma cells (U87), and human prostate cancer cells (PC3).

FIG. 2 is an illustration of an HSV-1 gKΔ31-68 mutant described herein, with (a) identifying a coiled coil peptide or Protein A IgG binding domain that replaces amino acid residues 31-68 of glycoprotein K; and (b) identifying a coiled coil peptide or antibody Fc domain that is fused to a polypeptide or antibody that binds to a specific tumor cell surface protein (such polypeptide or antibody is identified as (c) in FIG. 2).

FIG. 3 is a map showing the modified portion of a HSV genome (which corresponds to SEQ ID NO:11), particularly showing the location and orientation of the deletion of amino acid residues 31-68 of glycoprotein K and insertion of ARR2PB and Rat FGF 5′ UTR upstream of ICP27 (as further described and defined in Example-2).

FIG. 4 is a map showing the modified portion of another HSV genome (which corresponds to SEQ ID NO:9), particularly showing the “ΔgK-Au-TF-Fc-h1215” construct, with an inserted PD-L1 blocker, as further described and defined in Example-2.

FIG. 5 is a map showing the modified portion of another HSV genome (which corresponds to SEQ ID NO:10), particularly showing the “ΔgK-Au-TF-Fc-h1215” construct, which includes a modified TR region, as further described and defined in Example-2.

FIG. 6 is a schematic of an exemplary oncolytic HSV vector ΔgK-Au-TF-Fc-h1215.

FIG. 7 is a bar graph showing expression results of IL-12 for the HSV-1 gKΔ31-68 mutants described herein, within LNCaP and UMUC3 tumor cells, as described in Example-3.

FIG. 8 is a bar graph showing expression results of human IL-15/IL-15R-alpha complex for the HSV-1 gKΔ31-68 mutants described herein, within LNCaP and UMUC3 tumor cells, as described in Example-3.

FIG. 9 is a bar graph showing expression results of human IgG4 for the HSV-1 gKΔ31-68 mutants described herein, within LNCaP and UMUC3 tumor cells, as described in Example-3.

FIG. 10 is a line graph showing cytotoxicity of the HSV-1 gKΔ31-68 mutants described herein within LNCaP tumor cells.

FIG. 11 is a line graph showing cytotoxicity of the HSV-1 gKΔ31-68 mutants described herein within TRAMP-C2 tumor cells.

FIG. 12 is a line graph showing cytotoxicity of the HSV-1 gKΔ31-68 mutants described herein within UM-UC-3 tumor cells.

FIG. 13 is a line graph showing cytotoxicity of the HSV-1 gKΔ31-68 mutants described herein within MB-49-Luc tumor cells.

### BRIEF DESCRIPTION OF THE SEQUENCE LISTING

SEQ ID NO:1 represents an amino acid sequence of a wild type glycoprotein K of a human herpesvirus.

SEQ ID NO:2 represents an amino acid sequence of a modified glycoprotein K of a human herpesvirus, in which amino acid residues 31-68 thereof have been deleted (relative to the amino acid sequence of SEQ ID NO:1).

SEQ ID NO:3 represents an amino acid sequence of an E5 protein domain.

SEQ ID NO:4 represents an amino acid sequence of a K5 protein domain.

SEQ ID NO:5 represents an amino acid sequence of another coiled-coil peptide subunit, which is configured to bind to SEQ ID NO:6.

SEQ ID NO:6 represents an amino acid sequence of another coiled-coil peptide subunit, which is configured to bind to SEQ ID NO:5.

SEQ ID NO:7 represents an amino acid sequence of a Protein A IgG binding domain.

SEQ ID NO:8 represents an amino acid sequence of another Protein A IgG binding domain.

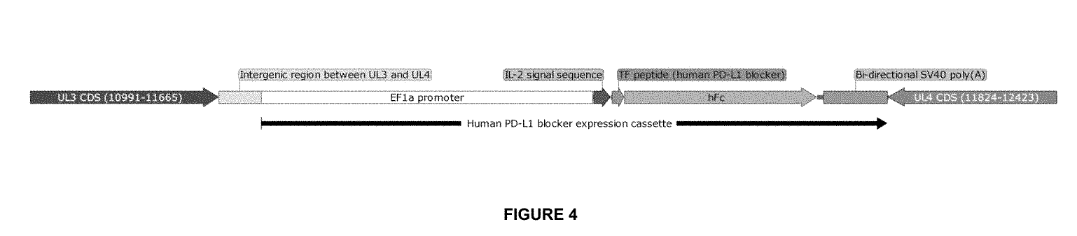

SEQ ID NO:9 represents a DNA sequence of the “ΔgK-Au-TF-Fc-h1215” construct, with an inserted PD-L1 blocker, as illustrated in FIG. 4.

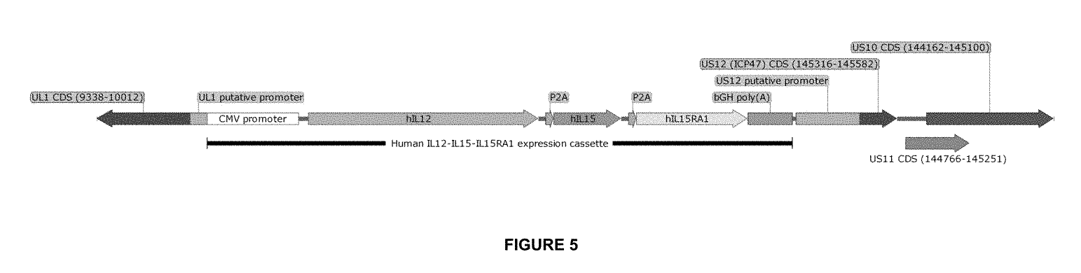

SEQ ID NO:10 represents a DNA sequence of the “ΔgK-Au-TF-Fc-h1215” construct, which includes a modified TR region, as illustrated in FIG. 5.

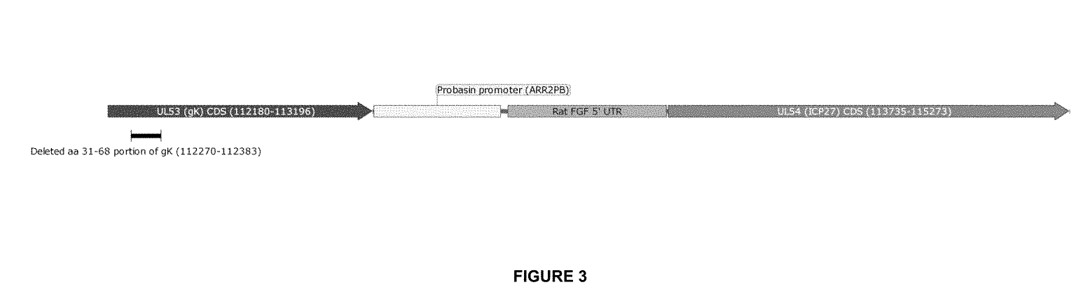

SEQ ID NO:11 represents a DNA sequence of a modified portion of a HSV genome that includes a deletion of amino acid residues 31-68 of glycoprotein K, with an insertion of ARR2PB and Rat FGF 5′ UTR upstream of ICP27, as illustrated in FIG. 3.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

### Definitions

As used herein, the following terms shall be accorded the meanings ascribed below.

The term “cancer,” as used herein, refers to a cancer of any kind and origin, including tumor-forming cells, blood cancers and/or transformed cells—but, in certain embodiments where expressly indicated, refers only to non-central nervous system (CNS) solid tumors.

The term “cancer cell,” as used herein, includes cancer or tumor-forming cells, transformed cells, or a cell that is susceptible to becoming a cancer or tumor-forming cell.

The term “oncolytic,” as used herein, refers to a tumor selective replicating virus, particularly a human herpesvirus, which induces cell death in the infected cancer cell and/or tissue. Although normal or non-tumor cells may be infected, cancer cells are infected and selectively undergo cell death, in comparison to the normal or non-cancer cells of a subject.

The term “cell death,” as used herein, includes all forms of cell death, including for example cell lysis and/or apoptosis.

The term “vector backbone,” as used herein, refers to a nucleic acid molecule that is used as a vehicle to deliver one or more nucleic acid molecules into a cell, e.g., to allow recombination. The vector backbone can refer optionally to a plasmid construct that is used to generate human herpesvirus or to a human herpesvirus genome (e.g., the non-recombined human herpesvirus genome). Optionally, the vector backbone is constructed to permit expression of one or more transgenes (e.g., an expression cassette) and the construct (e.g., vector backbone and transgene) can be referred to as an expression vector. A vector backbone into which has been inserted one or more nucleic acids to be transferred to a cell, may also be referred to as a “vector construct.”

The term “isolated vector construct,” as used herein, refers to a nucleic acid (a vector construct) that is substantially free of cellular material or culture medium when produced for example by recombinant DNA techniques.

The term “peptide,” as used herein, means a composition that is comprised of a chain of amino acid residues linked by peptide bonds. Unless otherwise indicated, the amino acid sequences for the peptides described herein are disclosed in the order from the amino terminus to the carboxyl terminus.

“E5 protein domain,” as used herein, means a coiled-coil peptide subunit, which exhibits a negative charge that is typically provided by a plurality of glutamic acid residues. A non-limiting example of an E5 protein domain is provided in SEQ ID NO:3.

“K5 protein domain,” as used herein, means a coiled-coil peptide subunit, which exhibits a positive charge that is typically provided by a plurality of lysine residues. A non-limiting example of a K5 protein domain is provided in SEQ ID NO:4.

The terms “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

The term “subject,” as used herein, includes all members of the animal kingdom including mammals, and suitably refers to humans.

The term “effective amount,” as used herein, means an amount of a therapeutic (i.e., the modified human herpesviruses or isolated vector constructs encoding the same) that is effective, at the necessary dosages and periods of treatment, to achieve a desired result. For example, in the context of treating a cancer, an effective amount is an amount that induces remission, reduces tumor burden, and/or prevents tumor spread or growth compared to the response obtained without administration of the modified and oncolytic human herpesviruses described herein. Effective amounts may vary according to factors such as the subject's disease state, age, gender, and weight, as well as the pharmaceutical formulation, the route of administration, and the like, but can nevertheless be routinely determined by one skilled in the art.

The HSV-1 gKΔ31-68 Mutants

According to certain preferred embodiments of the present invention, modified and isolated human herpesviruses are provided, which comprise a modified amino acid sequence that includes a deletion in the amino terminus of glycoprotein K (which otherwise forms a part of the wild type human herpesvirus). More particularly, in certain embodiments, the modified and oncolytic human herpesviruses comprise a modified glycoprotein K, in which amino acid residues 31-68 thereof have been deleted, relative to the wild type amino acid sequence of glycoprotein K (SEQ ID NO:1). In such embodiments, the modified and oncolytic human herpesviruses will comprise a modified glycoprotein K, which is represented by SEQ ID NO:2. In certain embodiments, the modified and oncolytic human herpesviruses will comprise a modified glycoprotein K, which is at least 95% identical to SEQ ID NO:2, or at least 98% identical to SEQ ID NO:2, or at least 99% identical to SEQ ID NO:2. Such modified and oncolytic human herpesviruses, along with the derivatives thereof disclosed herein, are also referred to herein as the “HSV-1 gKΔ31-68 mutants.” The invention provides that the modified and oncolytic human herpesviruses may comprise a modified (truncated) form of glycoprotein K, in both human herpesvirus type-1 and human herpesvirus type-2.

The invention further encompasses isolated vector constructs (as defined above), which encode for and will produce in an infected cell the HSV-1 gKΔ31-68 mutants described herein. In such embodiments, the deletion of amino acid residues 31-68 in the modified glycoprotein K region may also be described in terms of corresponding nucleotide positions. For example, in certain embodiments, the invention encompasses isolated vector constructs that encode for and produce the HSV-1 gKΔ31-68 mutants in an infected cell, with such constructs including a deletion of at least 114 nucleotides from a nucleic acid sequence that encodes the wild type glycoprotein K (with such deletion being positioned to delete the corresponding amino acid residues 31-68 from a wild type glycoprotein K region). As discussed further below, one or more exogenous genes may be positioned to replace the at least 114 nucleotides that would otherwise encode amino acid residues 31-68 of a wild type glycoprotein K. In addition, according to certain embodiments, the isolated vector constructs of the present invention may be designed to have the neurovirulence factor ICP34.5 gene (which is otherwise present in a wild type human herpesvirus) being deleted or functionally disabled. The invention provides that the isolated vector constructs that contain (1) a deletion of amino acid residues 31-68 from a wild type glycoprotein K region and (2) a deletion of the neurovirulence factor ICP34.5 gene are particularly useful for the treatment of central nervous system (CNS) tumors, as described further below; whereas, isolated vector constructs that contain a deletion of amino acid residues 31-68 from a wild type glycoprotein K region, and a wild type neurovirulence factor ICP34.5 gene, are more useful for the treatment of non-CNS tumors.

According to further embodiments, the HSV-1 gKΔ31-68 mutants of the present invention may further comprise an amino acid sequence encoded by one or more exogenous genes. For example, the amino acid sequence encoded by the one or more exogenous genes may provide a transport function (e.g., it may represent a targeting moiety or, alternatively, it may represent a type of linker that attaches a targeting moiety to the viral envelope), a protective function, or it may provide a therapeutic benefit to a subject. In the latter case, the amino acid sequence encoded by the one or more exogenous genes is preferably effective to (a) stimulate an immune system in a subject or (b) inhibit immune suppressors in a subject. In the first example (immune stimulators), the HSV-1 gKΔ31-68 mutants may be co-expressed or co-delivered with, for example, growth factors and cytokines that are effective to stimulate a subject's immune system (e.g., interleukins, such as IL12 or IL15; an IL15 receptor alpha unit; interferons; or combinations of the foregoing). In the second example (immune suppressor inhibitors), the HSV-1 gKΔ31-68 mutants may be co-expressed or co-delivered with, for example, proteins that block the activity of CTLA4 (which is expressed on the surface of activated cytotoxic T lymphocytes) or proteins that disrupt the interaction of PD-1 and proteins expressed on the surface of cancer cells known as PD-L1 and PD-L2. In certain embodiments, a suitable PD-L1 blocker that may be used in the HSV gKΔ31-68 mutants of the present invention is described in U.S. patent application Ser. No. 15/374,893, filed Dec. 9, 2016, which is hereby incorporated by reference in its entirety.

When one or more exogenous genes are included that provide a therapeutic benefit to a subject, such as in the examples above, the exogenous genes may positioned within a location of the viral genome that ensures suitable transcription and translation. When one or more exogenous genes are included to encode a type of linker that attaches a targeting moiety to the viral envelope, in that instance, the one or more exogenous genes will preferably replace the nucleotides that encode the amino terminus of glycoprotein K, e.g., the encoded linker will replace amino acid residues 31-68 of a wild type glycoprotein K (as described further below). The invention provides that multiple exogenous genes may be included in the HSV-1 gKΔ31-68 mutants, such as genes that (a) impart a therapeutic benefit to a subject and (b) encode a type of linker that attaches a targeting moiety to the viral envelope.

According to certain other embodiments of the present invention, the viral envelope of the modified oncolytic human herpesviruses may be decorated with an antibody, antibody fragment, or polypeptide that is capable of binding to a protein expressed on a cell surface of a cancer cell. In such embodiments, the oncolytic human herpesviruses will be designed to selectively target cancer cells, and to preferably avoid undesirable off-target effects and toxicity in a subject. In certain embodiments, the antibody, antibody fragment, or polypeptide may be tethered to the viral envelope through coiled-coil peptide interactions (as described further below). In other embodiments, when the viral envelope is decorated with an antibody (or certain antibody fragments), the antibody (or antibody fragments) may be tethered to the viral envelope through interactions between the Fc domain thereof and a Protein A IgG binding domain (as described further below).

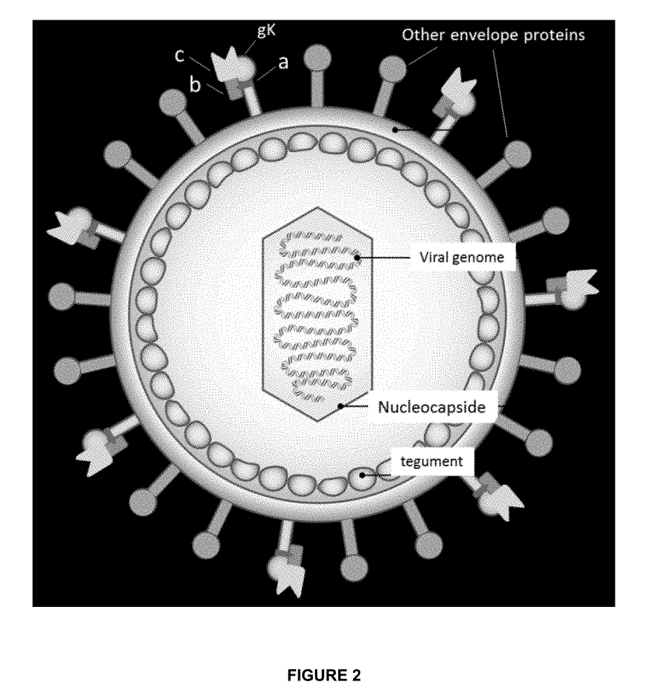

More particularly, according to these embodiments, the HSV-1 gKΔ31-68 mutants of the present invention may comprise a coiled-coil peptide subunit, which is designed to bind to another (corresponding) coiled-coil peptide subunit. More specifically, the HSV-1 gKΔ31-68 mutants may comprise an E5 protein domain or a K5 protein domain. In such embodiments, the amino terminus of glycoprotein K (which otherwise forms a part of the wild type human herpesvirus) is replaced with an E5 protein domain or a K5 protein domain, e.g., amino acid residues 31-68 from a wild type glycoprotein K region are replaced with an E5 protein domain or a K5 protein domain. In such embodiments, the negatively charged E5 protein domain (if included in the HSV-1 gKΔ31-68 mutant) will be chemically designed to bind, with high affinity, to a positively charged K5 protein domain. Likewise, the positively charged K5 protein domain (if included in the HSV-1 gKΔ31-68 mutant) will be chemically designed to bind, with high affinity, to a negatively charged E5 protein domain. In these embodiments, the HSV-1 gKΔ31-68 mutant will comprise a first coiled-coil peptide subunit (positioned at the amino terminus of glycoprotein K), e.g., an E5 protein, which will then bind to a corresponding second coiled-coil peptide subunit positioned within or fused to the antibody, antibody fragment, or polypeptide that is targeted to a cancer cell surface protein, to thereby tether the antibody, antibody fragment, or polypeptide to the viral envelope of the HSV-1 gKΔ31-68 mutant. This arrangement is further illustrated in FIG. 2.

A non-limiting example of an E5 protein domain is provided in SEQ ID NO:3, and a non-limiting example of a K5 protein domain is provided in SEQ ID NO:4. The E5 and K5 protein domains are comprised of a plurality of heptad units, i.e., a series of seven amino acid residues that are repeated a selected number of times. In the case of the E5 protein domain, the heptad unit of EVSALEK is repeated five times; whereas, in the case of the K5 protein domain, the heptad unit of KVSALKE is repeated five times. However, it will be appreciated that the coiled-coil peptides may comprise fewer or more heptad units. The invention provides that the coiled-coil peptides are preferably of similar size, and even more preferably, are the same size, with each ranging from about 21 to about 70 residues (3-10 heptad units) in length. SEQ ID NO:5 and SEQ ID NO:6, and derivatives thereof, represent additional non-limiting examples of coiled-coil peptide subunits that are designed to bind to each other.

According to yet further embodiments, the antibody, antibody fragment, or polypeptide that is targeted to a cancer cell surface protein may be tethered to the viral envelope of the HSV-1 gKΔ31-68 mutant via Fc domain/Protein A IgG binding domain interactions. More particularly, when the targeting moiety is an antibody (or an antibody fragment with a whole or partial Fc domain), the targeting moiety may be tethered to the viral envelope of the HSV-1 gKΔ31-68 mutant via binding to a Protein A IgG binding domain. In such embodiments, the amino terminus, e.g., amino acid residues 31-68 from a wild type glycoprotein K region, are replaced with a Protein A IgG binding domain, such as the domains represented by SEQ ID NO:7, SEQ ID NO:8, or derivatives thereof. This configuration is also illustrated in FIG. 2.

When the HSV-1 gKΔ31-68 mutant comprises a targeting moiety, the targeting moiety may recognize and bind to various proteins or other antigens expressed on the surface of cancer cells. For example, in certain embodiments, the targeting moiety may recognize and bind to EGFR, HER2, PSMA, CA-125, CEA, EpCAM, or derivatives of the foregoing. In other cases, the targeting moiety may recognize and bind to ALDH1, CD133, CD15, ABCG2, ABCB5, CD271, or derivatives of the foregoing.

According to further embodiments, the invention encompasses a composition comprising the HSV-1 gKΔ31-68 mutants, and/or the isolated vector constructs encoding the same, as described herein, which further include a pharmaceutically acceptable diluent or carrier. In certain embodiments, the diluent or carrier may comprise a phosphate-buffered saline solution. In other embodiments, the HSV-1 gKΔ31-68 mutants, and/or the isolated vector constructs encoding the same, may be combined or co-administered (either together or in series) with other chemotherapeutic agents.

According to yet further embodiments, the present invention includes methods of using the HSV-1 gKΔ31-68 mutants, and/or the isolated vector constructs encoding the same, described herein to prevent, treat, and/or ameliorate the effects of cancer. Such methods generally entail providing a subject with an effective amount of a HSV-1 gKΔ31-68 mutant described herein (and/or the isolated vector constructs encoding the same). The invention provides that such methods are particularly useful for preventing, treating, and/or ameliorating the effects of non-central nervous system (CNS) solid tumors. The invention provides that the effective amount of HSV-1 gKΔ31-68 mutants, and/or the isolated vector constructs encoding the same, to be administered to a subject will vary depending on various factors, such as the subject's disease state, age, gender, and weight, as well as the pharmaceutical formulation, the route of administration, and the like, but can nevertheless be routinely determined by one skilled in the art. In certain embodiments, the invention provides that the HSV-1 gKΔ31-68 mutants, and/or the isolated vector constructs encoding the same, will be administered to a subject via an intravenous route, an intraperitoneal route, or intratumorally.

### EXAMPLES

### Example-1

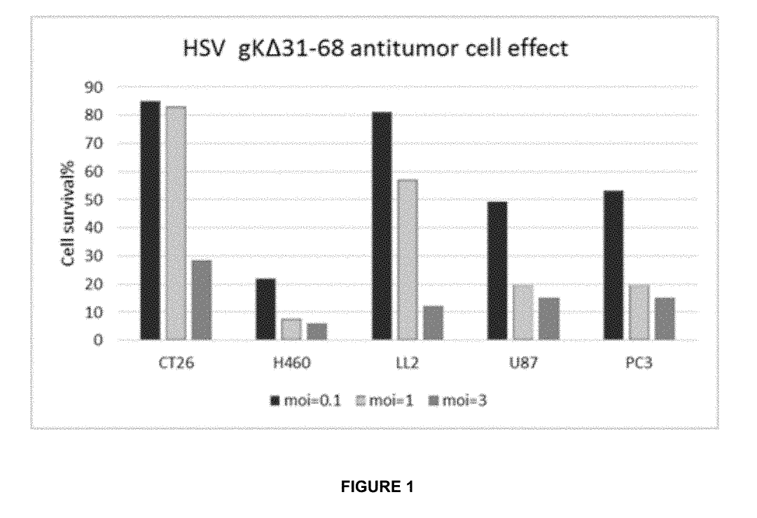

In this Example-1, various tumor cells were infected with the HSV-1 gKΔ31-68 mutant described herein for a period of 48 hours. The infected cancer cells included human colon cancer cells (CT26), human lung cancer cells (H460), mouse lung cancer cells (LL2), human glioma cells (U87), and human prostate cancer cells (PC3). Cell viability was measured and compared with control (mock infected) cells, at multiplicity of infection (MOI) points of 0.1, 1, and 3. As shown in FIG. 1, the presence of the HSV-1 gKΔ31-68 mutant exhibited a significant impact on the survival of each type of cancer cell tested and, more particularly, was able to achieve significant cell death among the cancer cells tested.

### Example-2

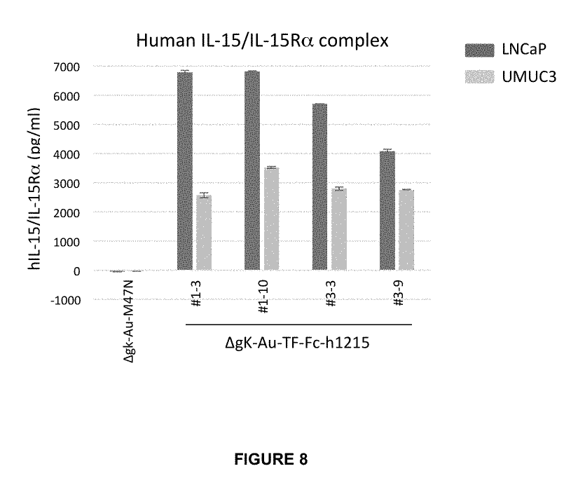

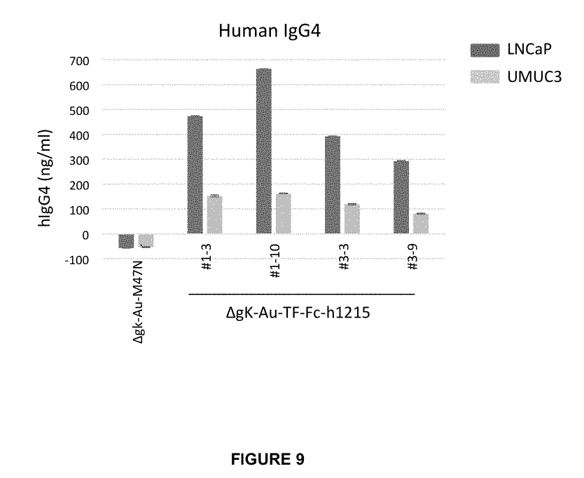

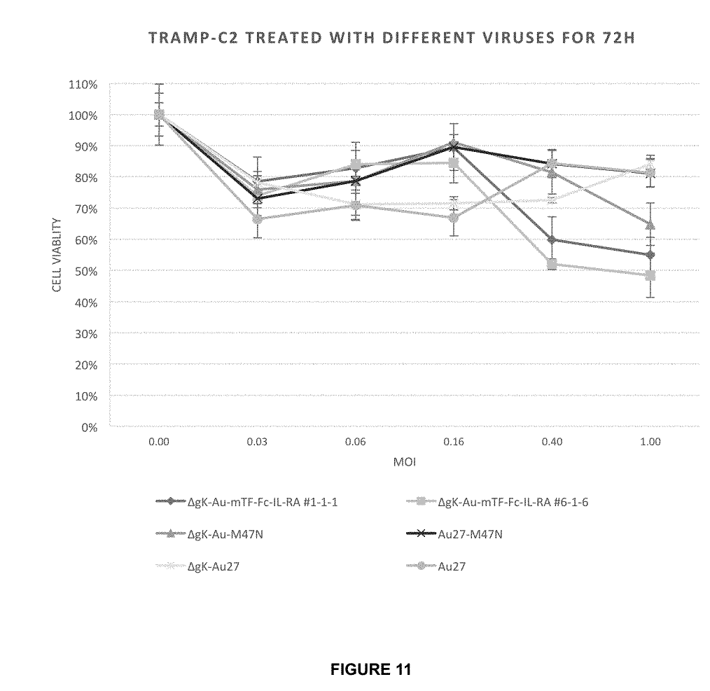

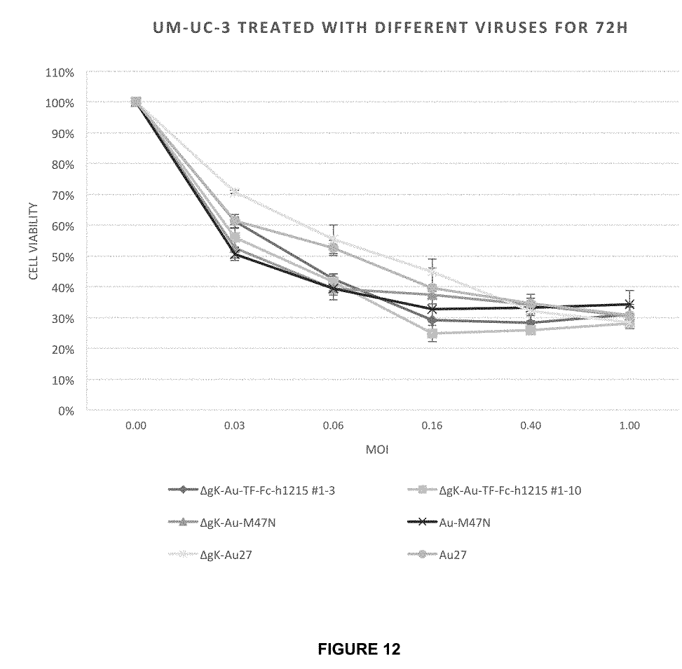

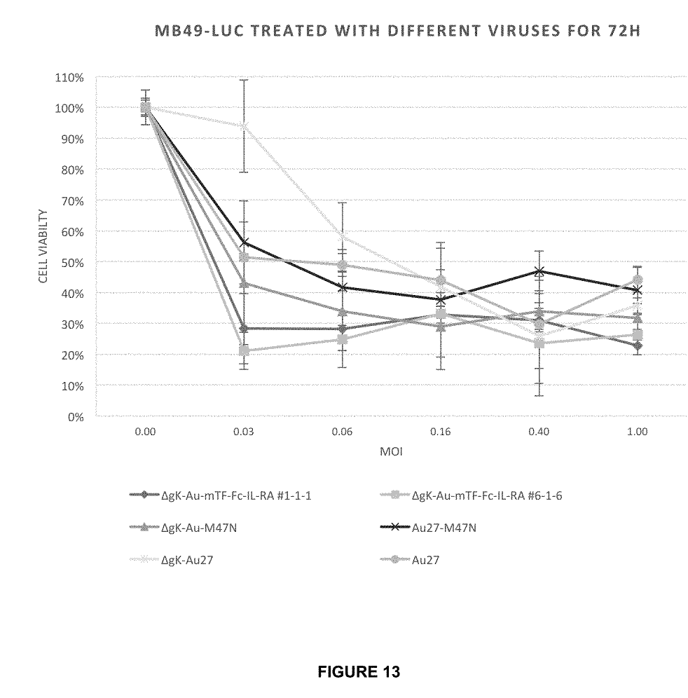

In this Example-2, multiple HSV-1 gKΔ31-68 mutants were constructed (which were subsequently tested for cytotoxic effects within various tumor cell lines, as further described in Example-4 below). FIGS. 3-6 provide maps that show the modified portions of the HSV genome in the HSV-1 gKΔ31-68 mutants. The following abbreviations, as used in FIGS. 3-13, have the meanings set forth below.

“TF-Fc” refers to a PD-L1 blocking peptide (TF) fused to an antibody Fc region.

“Au27” refers to a virus that includes a probasin promoter (ARR2PB) and a rat fibroblast growth factor (FGF) 5′ untranslated region (UTR) inserted within the intergenic region between UL53 (glycoprotein K) and UL54 (ICP27), which replaces the native promoter-regulatory region of UL54.

“ΔgK-Au27” refers to a virus that includes a modified UL54 promoter-regulatory region that is identical to the Au27 mutant and a deletion of the N-terminal amino acids 31-68 of UL53 (glycoprotein K).

“Au-M47N” refers to a virus that includes a modified UL54 promoter-regulatory region identical to the Au27 mutant and insertion of an exogenous promoter and poly(A) flanking an empty multiple cloning site (MCS) within the deleted terminal repeat (TR) region of the viral genome, which was subsequently used for insertion of an expression cassette encoding IL-12, IL-15, and IL-15 receptor alpha subunit. Notably, however, the invention provides that either the terminal or internal repeat region may be deleted (to create about 20 kb of space) and used for such purpose (i.e., for insertion of sequences that encode immune stimulating factors, as well as checkpoint inhibitors).

“ΔgK-Au-M47N” refers to a virus that includes a modified UL54 promoter-regulatory region identical to the Au27 mutant; a deletion of the N-terminal amino acids 31-68 of UL53 (glycoprotein K); and insertion of an exogenous promoter and poly(A) flanking an empty MCS within the deleted terminal repeat (TR) region of the viral genome that was subsequently used for insertion of an expression cassette encoding IL-12, IL-15, and IL-15 receptor alpha subunit.

“ΔgK-Au-mTF-Fc-IL-RA” refers to a virus that includes a modified UL54 promoter-regulatory region identical to the Au27 mutant; a deletion of the N-terminal amino acids 31-68 of UL53 (glycoprotein K); an insertion of a mouse PD-L1 blocker within the intergenic region between UL3 and UL4; and a modified terminal repeat (TR) region carrying an expression cassette encoding mouse IL-12, human IL-15, and human IL-15 receptor alpha subunit.

“ΔgK-Au-TF-Fc-h1215” refers to a virus that includes a modified UL54 promoter-regulatory region identical to the Au27 mutant; a deletion of the N-terminal amino acids 31-68 of UL53 (glycoprotein K); an insertion of a PD-L1 blocker within the intergenic region between UL3 and UL4; and a modified terminal repeat (TR) region carrying an expression cassette encoding human IL-12, human IL-15, and human IL-15 receptor alpha subunit. Notably, in these Examples, the intergenic region between UL3 and UL4 was used to insert the PD-L1 blocker cassette; however, such location may also be used for the sequences that encode immune stimulating factors and checkpoint inhibitors.

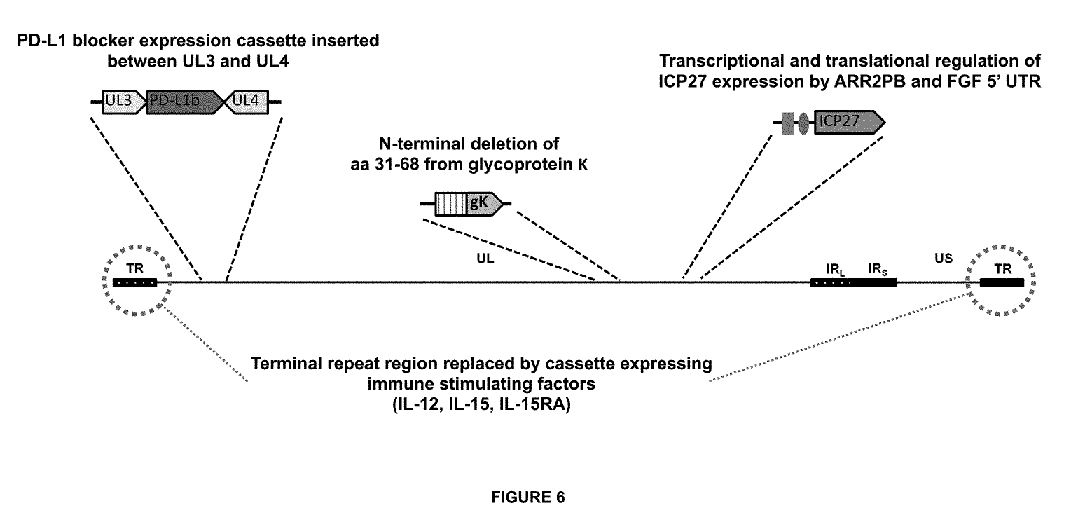

FIGS. 3-6 show maps/schematics of various modifications that were made to the same HSV genome (ΔgK-Au-TF-Fc-h1215). Specifically, FIG. 3 provides a map showing the modified portion of a HSV genome (which corresponds to SEQ ID NO:11), particularly showing the location and orientation of the deletion of amino acid residues 31-68 of glycoprotein K and insertion of ARR2PB and Rat FGF 5′ UTR upstream of ICP27. FIG. 4 provides a map showing the modified portion of another HSV genome (which corresponds to SEQ ID NO:9), particularly showing the “ΔgK-Au-TF-Fc-h1215” construct, with an inserted PD-L1 blocker. FIG. 5 provides a map showing the modified portion of yet another HSV genome (which corresponds to SEQ ID NO:10), particularly showing the “ΔgK-Au-TF-Fc-h1215” construct, which includes a modified TR region. FIG. 6 provides a map showing an exemplary ΔgK-Au-TF-Fc-h1215, as such HSV vector was described above. The invention may, optionally, further encompass and employ the use of certain HSV mutants that are described within PCT Application No. PCT/US17/30308, filed Apr. 29, 2017, which is incorporated herein by reference in its entirety.

### Example-3

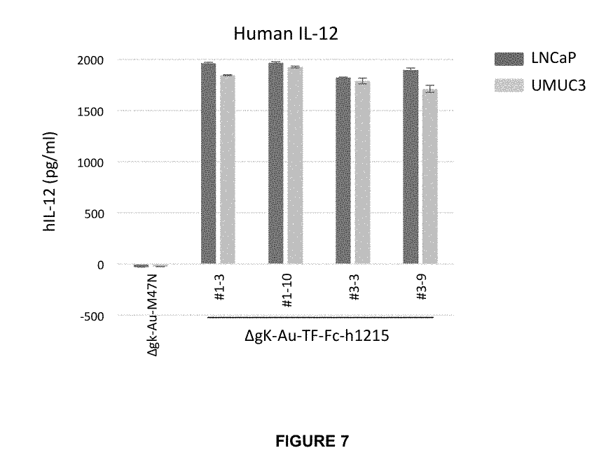

In this Example-3, expression of IL-12, IL-15, and PD-L1 blocker for the HSV-1 gKΔ31-68 mutants described in Example-2 was measured. Notably, in this Example-3, the PD-L1 blocker was specifically designed for use in human subjects, as opposed to the mouse PD-L1 blocker that was present within ΔgK-Au-mTF-Fc-IL-RA. In this Example-3, 3×10⁴ LNCaP or UMUC3 tumor cells were seeded into each well of a 96-well plate and cultured at 37° C. overnight. The next day, seeded cells were infected with ΔgK-Au-M47N backbone, or different clones of ΔgK-Au-TF-Fc-h1215 virus (MOI=1), for 48 hours and the production of human IL-12, human IL-15/IL-15R-alpha complex, and human IgG4 was assessed. The expression results are summarized in FIGS. 7-9.

### Example-4

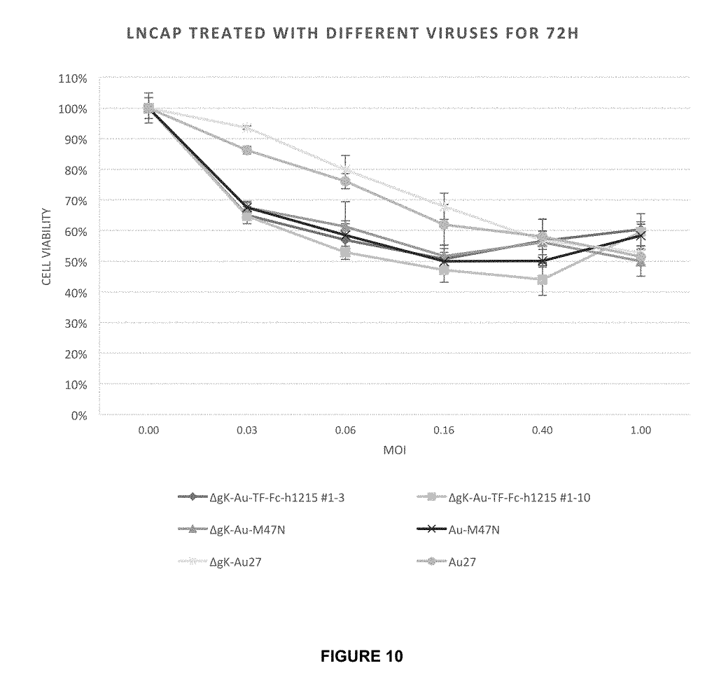

In this Example-4, cytotoxicity of the HSV-1 gKΔ31-68 mutants described in Example-2 was measured against a panel of different tumor cell lines. Specifically, LNCaP, TRAMP-C2, UM-UC-3, and MB-49-Luc tumor cells were infected with HSV-1 gKΔ31-68 mutants (as indicated in FIGS. 10-13) at MOI=0, 0.03, 0.06, 0.16, 0.40, or 1.00. In these mutants, the PD-L1 blocker was fused to Fc, which enabled detection using an anti-IgG4 antibody. Cell viability was subsequently quantified using an MTT assay at 72 hours post-infection. The cytotoxicity results are summarized in FIGS. 10-13.

The many aspects and benefits of the invention are apparent from the detailed description, and thus, it is intended for the following claims to cover all such aspects and benefits of the invention that fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A modified and isolated human herpesvirus, which comprises a modified amino acid sequence that includes a deletion in a region that represents an amino terminus of glycoprotein K.

2. The modified and isolated human herpesvirus of claim 1, wherein the modified amino acid sequence comprises an amino acid sequence that is at least 98% identical to SEQ ID NO:2.

3. The modified and isolated human herpesvirus of claim 2, which is a human herpesvirus type-1 or type-2.

4. The modified and isolated human herpesvirus of claim 3, which further comprises an amino acid sequence encoded by one or more exogenous genes.

5. The modified and isolated human herpesvirus of claim 4, wherein the amino acid sequence encoded by the one or more exogenous genes provides a therapeutic benefit to a subject.

6. The modified and isolated human herpesvirus of claim 5, wherein the amino acid sequence encoded by the one or more exogenous genes is effective to (a) stimulate an immune system in a subject or (b) inhibit immune suppressors in a subject.

7. The modified and isolated human herpesvirus of claim 6, wherein the amino acid sequence encoded by the one or more exogenous genes represents interleukin-12, interleukin-15, an interleukin-15 receptor alpha unit, an interferon, or combinations of the foregoing.

8. The modified and isolated human herpesvirus of claim 6, wherein the amino acid sequence encoded by the one or more exogenous genes represents an agent that is effective to block or inhibit immune suppressors.

9. The modified and isolated human herpesvirus of claim 8, wherein the agent is effective to disrupt interactions between (a) PD-1 and (b) PD-L1 or PD-L2.

10. The modified and isolated human herpesvirus of claim 1, wherein a ICP34.5 gene of a wild type human herpesvirus is either deleted or functionally disabled.

11. An isolated vector construct that encodes a modified and isolated human herpesvirus of claim 1.

12. A method of treating cancer, including non-central nervous system solid tumors, which comprises providing a subject with an effective amount of a modified and isolated human herpesvirus of claim 1 within a suitable diluent or carrier, wherein the modified and isolated human herpesvirus is delivered to the subject via an intravenous route, an intraperitoneal route, or intratumorally, wherein the modified and isolated human herpesvirus may optionally be provided to the subject in combination with chemotherapy or a radiation treatment.
